(19) United States
(12) Patent Application Publication
(10) Pub. No.: US 2003/0013640 A1
(43) Pub. Date: Jan. 16, 2003

(54) Integrin linked kinase modulation of leukocyte trafficking

(76) Inventors: Ljiljana Kojic, Vancouver (CA); Gabe Kalmar, Richmond (CA); David M. Moran, Woking (GB)

Correspondence Address: BOZICEVIC, FIELD & FRANCIS LLP, 200 MIDDLEFIELD RD, SUITE 200, MENLO PARK, CA, 94025, US

(21) Appl. No.: 10/163,385

(22) Filed: Jun. 4, 2002

Related U.S. Application Data
(60) Provisional application No. 60/296,262, filed on Jun. 5, 2001.

(51) Int. Cl. A61K 38/00

(52) U.S. Cl. 514/2

(57) ABSTRACT

Methods are provided to specifically modulate the trafficking of leukocytes that signal through the integrin linked kinase. Agonists or other agents that enhance ILK function increase leukocyte accumulation at a targeted site. In an alternative embodiment, the agent is an antagonist that blocks ILK biological activity and decreases leukocyte accumulation. The methods of the invention manipulate the integrin mediated signaling during trafficking to affect the localization of immune effector cells in targeted tissues.

### BACKGROUND OF THE INVENTION

Most mature leukocytes continuously circulate between the blood and lymphatic organs. Leukocytes leave the blood by recognizing and binding to the vascular endothelial cells. Thereafter, they migrate between the endothelial cells into the surrounding tissues. Leukocyte trafficking allows the full repertoire of immunological specificities to be available for immune reactions throughout the body, and it also facilitates the cell-cell interactions required for the generation and control of immune responses. Trafficking also controls the location of leukocytes during development and differentiation, for example in the movement of lymphocytes from the bone marrow to the thymus, the bone marrow homing of stem cells, etc.

Leukocyte adherence to endothelial cells is dependent on interactions between complementary adhesion molecules expressed on both cell types. Normally, this binding is to specialized postcapillary venules called high endothelial venules (HEV). Functionally distinct leukocyte-HEV recognition systems mediating migration to peripheral lymph nodes, mucosal lymphoid organs, synovium, skin and lung-associated lymphoid tissues in an organ-specific manner have been described.

The process of trafficking, (or homing) is believed to consist of a number of discrete steps, including migration of cells to a targeted tissue, e.g. by chemotaxis. The leukocytes roll along the endothelium, until delivery of a triggering signal that activates leukocyte integrins. The integrin activation step results in establishment of strong adhesion to the endothelium. Shortly after adhesion, the leukocytes enter the extra-vascular space though endothelial cell-cell junctions.

The integrins are a family of alpha, beta heterodimeric receptors that mediate dynamic linkages between extracellular adhesion molecules and the intracellular actin cytoskeleton. Integrins are expressed by all multicellular animals, but their diversity varies widely among species; for example, in mammals, 19 alpha and 8 beta subunit genes encode polypeptides that combine to form 25 different receptors. Gene deletion has demonstrated essential roles for almost all integrins, with the defects suggesting widespread contributions to both the maintenance of tissue integrity and the promotion of cellular migration. Animal-model studies have also shown that integrins contribute to the progression of many common diseases, and have implicated them as potential therapeutic targets.

Integrins play important roles in the process of leukocyte homing. In one step of the leukocyte-endothelium interaction model, there is a rapid activation of integrins on leukocytes. This activation is mediated by different molecules, and permits the arrest of the rolling leukocyte. For example, the ligand interaction of L-selectin, or antibody cross-linking, leads to partial activation of leukocyte β2 integrins by a signal transduction pathway involving mitogen-activated protein kinases. Fast signaling may also be mediated by soluble factors associated with the endothelial surface, including chemoattractants and the chemokine family. The passage through the perivascular space may also involve integrin interactions.

Interactions between leukocyte surface receptors and their ligands on vascular endothelial cells critically control lymphocyte traffic between the blood and various lymphoid organs, as well as extravasation of leukocytes into sites of inflammation. In view of the importance of leukocyte trafficking in immune responses, the further characterization and manipulation of the pathways regulating trafficking are of great interest.

### SUMMARY OF THE INVENTION

Methods and compositions are provided to modulate the trafficking of leukocytes to specific extravascular sites. The trafficking of leukocytes is prevented by the administration of ILK blocking agents; compounds that otherwise prevent the binding of natural ILK ligands to ILK; or compounds that prevent expression of, or signaling through, ILK. Compounds that enhance ILK activity increase the trafficking of leukocytes to targeted sites. The modulation of trafficking is used to regulate immune processes at targeted sites, for example to decrease undesirable inflammatory, or atopic and allergic responses.

### DETAILED DESCRIPTION OF THE EMBODIMENT

In the subject methods, compounds that modulate the triggering activity of ILK are administered systemically or locally to alter the trafficking behavior of leukocytes. Trafficking, or homing, is used herein to refer to the biological activities and pathways that control the localization of leukocytes in a mammalian host. Such trafficking may be associated with disease, e.g. inflammation, allergic reactions, etc., or may be part of normal biological homeostasis.

Local administration that provides for a prolonged localized concentration, which may utilize sustained release implants or other topical formulation, is of particular interest. In one embodiment of the invention the trigger modulating compound is an agonist of ILK, which acts to enhance the triggering effect. In an alternative embodiment, the trigger modulating compound blocks ILK activity. In vivo uses of the method are of interest for therapeutic and investigational purposes. In vitro uses are of interest for drug screening, determination of physiological pathways, and the like.

ILK is a mediator of signaling through an integrin receptor, and can link the external binding of integrins to intracellular pathways. Integrin activation during leukocyte trafficking is associated with responsiveness to adhesion, and is also related to such events as chemokine binding. Integrin activation results in alterations of cell adhesion, structure and extravasation. By intervening at this point of the integrin signaling pathway, the physiological localization of immune effector and regulatory cells can be altered, thereby providing therapeutic benefit to a patient.

Cells of interest for the present methods include the polymorphonuclear cells, e.g. basophils, eosinophils, and neutrophils. One aspect of the invention is the effect of modulating polymorphonuclear leukocytes (PMN) trafficking. The α2β1 (VLA-2) integrin is used by PMNs for locomotion in extravascular tissue. The integrin is upregulated in extravasated PMN, and absent from circulating blood PMN (Werr et al. (2000) Blood 95(5):1804-9). The administration of agents that block ILK decreases the trafficking of PMN to sites of inflammation, and the administration of ILK activating agents may enhance PMN trafficking.

PMNs include neutrophils, which are primarily found in storage in the bone marrow. The major inflammatory functions of neutrophils include phagocytosis and secretion of pro-inflammatory substances. As a general rule, neutrophils are the predominant cell type in acute inflammation. Pro-inflammatory substances released by neutrophils include lysosomal enzymes, products of oxygen metabolism, and products of arachidonic acid metabolism.

Eosinophils are prominent at sites of allergic reactions, and with parasitic infections. Eosinophil secretory products inactivate many of the chemical mediators of inflammation. This phenomenon is most obvious with mast cell-derived mediators. Mast cells produce a chemotactic factor for eosinophils. Secretory products of eosinophils can kill parasitic larvae by disrupting their cuticles, and parasite-induced IgE-containing immune complexes are chemotactic for eosinophils.

Basophils are the circulating counterpart of mast cells, and are often associated with allergic reactions and parasitic infections. Their major inflammatory function is release of basophil granule contents that incite vascular changes at sites of acute inflammation. Increased numbers of basophils are located in skin affected with ectoparasites.

Mononuclear cells are also of interest, including mononuclear phagocytes and mast cells. Another aspect of the invention is the modulation of monocyte trafficking. The monocyte fibronectin receptor is α5β1 integrin. Changes in expression of this integrin result in alteration of monocyte migration into tissues. Binding of monocytes through this integrin receptor modulates adhesive interactions, and causes monocytes to accumulate. Monocytes are of interest as immune effector cells, and as antigen presenting cells. The administration of agents that block ILK decreases the trafficking of monocytes to sites of inflammation, and the administration of ILK activating agents may enhance monocyte trafficking.

The mononuclear phagocyte system is comprised of both circulating and fixed populations of cells. The circulating component is the monocyte. Upon migration into tissues these are referred to as histiocytes or tissue macrophages. The major fixed macrophages include: Sinusoidal lining cells of the spleen, lymph nodes, liver, and bone marrow; connective tissue histiocytes; mobile macrophages on serosal surfaces; alveolar macrophages within the lung; microglia in the nervous system; and mesangial macrophages within renal glomeruli. Macrophages produce a variety of substances that are involved in inflammation. Mast cells are important mediators of certain allergic reactions. Mast cell membranes have abundant IgE receptor sites, anywhere from 30,000 to 500,000 per cell. If a particular antigen incites an IgE response, the resulting IgE is bound to the IgE receptors on mast cell surfaces via the Fc portion of the immunoglobulin molecule. Interaction of an antigen with surface-bound IgE results in cross-linking of the IgE molecules, mast cell activation, and ultimately mast cell degranulation.

### Integrin Linked Kinase

The integrin linked kinase, ILK, binds to the cytoplasmic tail of β1 and β3 integrins, thereby regulating outside-in and inside-out signaling. ILK has been implicated in regulation of cell adhesion and fibronectin matrix assembly, and is involved in the trafficking of leukocytes. The genetic sequence of human ILK is disclosed in U.S. Pat. Nos. 6,013,782; and 6,001,622, herein incorporated by reference. ILK is a serine threonine kinase having two functional domains, identified by comparison of the ILK sequence against those found in current protein databases. These are the catalytic domain, responsible for phosphotransferase activity (kinase domain), and a non-overlapping domain in the amino terminus, comprised of four contiguous ankyrin-like repeats. The function of ankyrin repeats in ILK is to mediate protein-protein interactions, although the ankyrin repeat domain is not required for the binding of ILK to integrin. ILK bridges integrin in the plasma membrane with intracellular proteins active regulating the cell's response to ECM signals.

ILK regulates integrin extracellular activity (ECM interactions) from inside the cell via its direct interaction with the integrin subunit (colloquially known as inside-out signaling). Interfering with ILK activity allows the specific targeting of integrin function, while leaving other essential signaling pathways intact.

ILK activity can be stimulated by phosphatidylinositol 3,4,5 trisphosphate in vitro. Both insulin and fibronectin can rapidly stimulate ILK activity in a phosphoinositide-3OH kinase (PI(3)K)-dependent manner. In addition, constitutively active PI(3)K activates ILK. The activated ILK can then inhibit the activity of glycogen synthase kinase-3 (GSK-3), contributing to ILK induced nuclear translocation of β-catenin. ILK can also phosphorylate protein kinase B (PKB/AKT) on serine-473, resulting in its activation, demonstrating that ILK is involved in agonist stimulated PI(3)K-dependent PKB/AKT activation.

The integrins are a family of heterodimeric membrane glycoproteins that function as major receptors for cell adhesion molecules and extracellular matrix proteins. All integrins consist of two non-covalently associated subunits, α and β. The β1 subunit (which is also referred to as CD29, very late activation protein, or VLA-B) associates with a number of different α chains, including α1, 2, 3, 4, 5, 6 and 7. VLA-4 (α4β1 integrin) is the receptor for fibronectin. The VLA family includes VLA-1, VLA-2, VLA-3, VLA-4, VLA-5 and VLA-6, which are heterodimers comprising β1 integrin and the different α chains. Several of the VLA proteins are expressed on activated T cells. VLA-4 in particular plays an important role in mediating mononuclear leukocyte trafficking during inflammation (see, e.g. Hemler (1990 Immunol. Rev. 114:45-65).

### ILK Modulating Agents

Agents that block ILK activity provide a point of intervention in an important signaling pathway. Numerous agents are useful in reducing ILK activity, including agents that directly modulate ILK expression, e.g. expression vectors, anti-sense specific for ILK, ILK specific antibodies and analogs thereof, small organic molecules that block ILK catalytic activity, etc.; and agents that affect ILK activity through direct or indirect modulation of [Ptdlns(3,4,5)P₃] levels in a cell. For example, small molecule inhibitors of integrin linked kinase are described in U.S. Pat. No. 6,214,813. Antisense inhibitors of ILK are described in U.S. Pat. No. 6,177,273, each herein incorporated by reference.

ILK modulating agents are molecules that specifically act as an agonist to enhance ILK biological activity; or that act as antagonists that block ILK biological activity, for example the interaction between ILK and its ligands. Such agents may activate the molecule through the ligand binding site, block the ligand binding site, etc. Blocking agents do not activate ILK triggering of adhesion. Agonists may activate integrin, or enhance the triggering activity of ILK.

Agents of interest for down-regulating ILK activity include direct blocking of [Ptdlns(3,4,5)P₃] binding sites through competitive binding, steric hindrance, etc. Of particular interest are antibodies that bind to the PH domains, thereby blocking the site. Antibodies include fragments, e.g. F(Ab), F(Ab)′, and other mimetics of the binding site. Such antibodies can be raised by immunization with the protein or the specific domain. Mimetics are identified by screening methods, as described herein. Analogs of [Ptdlns(3,4,5)P₃] that compete for binding sites but do not result in activation of ILK are also of interest.

ILK activity is upregulated by the presence of the lipid [Ptdlns(3,4,5)P₃]. The activity of. ILK is manipulated by agents that affect cellular levels of [Ptdlns(3,4,5)P₃], or that block the binding of [Ptdlns(3,4,5)P₃] to ILK. This lipid binds to specific amino acid residues in ILK. The amino acid sequence of ILK contains a sequence motif found in pleckstrin homology (PH) domains, which are involved in the binding of phosphatidylinositol phosphates. The activity of. ILK is also down-regulated by inhibiting the activity of PI(3) kinase, thereby decreasing cellular levels of [Ptdlns(3,4,5)P₃].

Drug screening can be used to identify agents that modulate ILK function. One can identify ligands or substrates that bind to, modulate or mimic the action of ILK. A wide variety of assays may be used for this purpose, including labeled in vitro protein-protein binding assays, electrophoretic mobility shift assays, immunoassays for protein binding, and the like. Knowledge of the 3-dimensional structure of ILK, derived from crystallization of purified recombinant ILK protein, leads to the rational design of small drugs that specifically inhibit ILK activity. These drugs may be directed at specific domains of ILK, e.g. the kinase catalytic domain, ankyrin repeat domains, pleckstrin homology domains, etc. Among the agents of interest for drug screening are those that interfere with the binding of cytoplasmic integrin tails to ILK; the kinase activity of ILK; binding of [Ptdlns(3,4,5)P₃] to the PH domains of ILK and agents that inhibit the production of [Ptdlns(3,4,5)P₃] by PI(3) kinase.

The term “agent” as used herein describes any molecule, e.g. protein or pharmaceutical, with the capability of altering or mimicking the physiological function of ILK. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Assays of interest may detect agents that mimic or block ILK function, such integrin binding, kinase activity, down regulation of E-cadherin, up regulation of LEF-1, binding properties, etc. For example, an expression construct comprising a ILK gene may be introduced into a cell line under conditions that allow expression. The level of ILK activity is determined by a functional assay, as previously described. In one screening assay, candidate agents are added, and the formation of fibronectin matrix is detected. In another assay, the ability of candidate agents to enhance ILK function is determined.

Agents of interest include inhibitors of PI(3) kinase, e.g. wortmannin, LY294002, etc. Physiologically effective levels of wortmannin range from about 10 to 1000 nM, usually from about 100 to 500 nM, and optimally at about 200 nM. Physiologically effective levels of LY294002 range from about 1 to 500 μM, usually from about 25 to 100 μM, and optimally at about 50 μM. The inhibitors are administered in vivo or in vitro at a dose sufficient to provide for these concentrations in the target tissue.

### Methods of Treatment

An ILK blocking agent prevents triggering of integrin mediated adhesion, and is useful in the inhibition of graft rejection or other undesirable immune responses by preventing the accumulation of effector cells at the site of graft implantation or inflammation. Such effector cells may include T helper and killer cell subsets, monocytes and other antigen presenting cells, PMN, etc. For example, the methods can prevent intra-islet infiltration by effector cells to inhibit development of insulin-dependent diabetes mellitus; blocking infiltration of effector cells into the central nervous system to treat multiple sclerosis and other demyelinating diseases; blocking the accumulation of effector cells in the synovial joints of patients suffering from rheumatoid arthritis; accumulation of effector cells to influence immune responsiveness, and the like.

ILK agonists are useful in enhancing the immune reaction at a targeted site. For example, in burn patients it may be desirable to prophylactically increase the effector cell population at the burn sites. Other infections, particularly localized infections, may be treated this way, including, without limitation, human herpes viruses including herpes simplex viruses (HSV) types 1 and 2, Epstein Barr virus (EBV), cytomegalovirus (CMV), varicella zoster virus (VZV) and human herpes virus 6 (HHV-6), particularly infections of the mouth and genitals, hepatitis B virus (HBV) and hepatitis C virus (HCC) infections of the liver, etc.

The effect of a treatment can be monitored by determining the localization of different leukocyte subsets. Leukocyte subsets, e.g. T cells including Th1, Th2, Th3, regulatory T cells and cytotoxic T cells; polymorphonuclear cells (PMN); monocytes and macrophages; dendritic cells; B cells; and the like, are characterized according to the cell surface expression of certain known antigens. Verification of the identity of the cells of interest may be performed by any convenient method, including antibody staining and analysis by fluorescence detection, ELISA, etc., reverse transcriptase PCR, transcriptional amplification and hybridization to nucleic acid microarrays, etc.

### Conditions of Interest

Conditions of inflammation-associated or allergic reaction patterns of the skin include atopic dermatitis or infantile eczema; contact dermatitis, psoriasis, lichen planus; hypersensitivity or destructive responses to infectious agents, etc. Such diseases benefit from the administration of ILK agonists. The treatment decreases the number of effector immune cells at the sites of inflammation.

Rheumatoid arthritis (RA) is a chronic autoimmune inflammatory synovitis affecting 0.8% of the world population. Current therapy for RA utilizes therapeutic agents that non-specifically suppress or modulate immune function. Such therapeutics, including the recently developed TNFα antagonists, are not fundamentally curative, and disease activity rapidly returns following discontinuation of therapy. The ability of the compounds of this invention to treat arthritis can be demonstrated in a murine collagen-induced arthritis model according to the method of Kakimoto, et al., Cell Immunol 142: 326-337, 1992, in a rat collagen-induced arthritis model according to the method of Knoerzer, et al., Toxicol Pathol 25:13-19, 1997, in a rat adjuvant arthritis model according to the method of Halloran, et al., Arthitis Rheum 39: 810-819, 1996, in a rat streptococcal cell wall-induced arthritis model according to the method of Schimmer, et al., J. Immunol 160: 1466-1477, 1998, or in a SCID-mouse human rheumatoid arthritis model according to the method of Oppenheimer-Marks et al., J Clin Invest 101: 1261-1272, 1998.

Degenerative joint diseases may be inflammatory, as with seronegative spondylarthropathies, e.g. ankylosing spondylitis and reactive arthritis; rheumatoid arthritis; gout; and systemic lupus erythematosus. There is significant immunological activity within the synovium during the course of inflammatory arthritis. While treatment during early stages is desirable, the adverse symptoms of the disease may be at least partially alleviated by treatment during later stages. The ability of the compounds of this invention to treat Lyme arthritis can be demonstrated according to the method of Gross et al., Science 281, 703-706, 1998.

A quantitative increase in myelin-autoreactive T cells with the capacity to secrete IFN-gamma is associated with the pathogenesis of MS and EAE, suggesting that autoimmune inducer/helper T lymphocytes in the peripheral blood of MS patients may initiate and/or regulate the demyelination process in patients with MS. The overt disease is associated with muscle weakness, loss of abdominal reflexes, visual defects and paresthesias. During the presymptomatic period there is infiltration of leukocytes into the cerebrospinal fluid, inflammation and demyelination.

Human IDDM is a cell-mediated autoimmune disorder leading to destruction of insulin-secreting beta cells and overt hyperglycemia. T lymphocytes invade the islets of Langerhans, and specifically destroy insulin-producing β-cells. The depletion of β cells results in an inability to regulate levels of glucose in the blood. An increase in the number of T lymphocytes in the pancreas, islet cell antibodies and blood glucose is indicative of the disease. After the onset of overt diabetes, patients with residual beta cell function, evidenced by the plasma persistence of insulin C-peptide, may benefit from the subject treatment, to prevent further loss of function. The ability of compounds of this invention to treat autoimmune diabetes can be demonstrated in an NOD mouse model according to the method of Hasagawa et al., Int Immunol 6:831-838, 1994, or in a murine streptozotocin-induced diabetes model according to the method of Herrold et al., Cell Immunol 157:489-500,1994.

Other inflammatory conditions of interest include inflammatory bowel disease, lung disease, etc. The ability of compounds of this invention to treat inflammatory lung injury can be demonstrated in a murine oxygen-induced lung injury model according to the method of Wegner et al., Lung 170:267-279, 1992, in a murine immune complex-induced lung injury model according to the method of Mulligan et al., J Immunol 154:1350-1363, 1995, or in a murine acid-induced lung injury model according to the method of Nagase et al., Am J Respir Crit Care Med 154:504-510, 1996. The ability of compounds of this invention to treat inflammatory bowel disease can be demonstrated in a rabbit chemical-induced colitis model according to the method of Bennet et al., J Pharmacol Exp Ther 280:988-1000, 1997.

Allergy, or atopy is an increased tendency to IgE-based sensitivity resulting in production of specific IgE antibody to an immunogen, particularly to common environmental allergens such as insect venom, house dust mite, pollens, molds or animal danders. Allergic responses are antigen specific. The immune response to the antigen is further characterized by the over-production of Th2-type cytokines, e.g. IL-4, IL-5 and IL-10, by the responding T cells. The sensitization occurs in genetically predisposed people after exposure to low concentrations of allergen; cigarette smoke and viral infections may assist in the sensitization process.

Included in the group of patients suffering from atopy are those with asthma associated allergies. About 40% of the population is atopic, and about half of this group develop clinical disease ranging from trivial rhinitis to life-threatening asthma. After sensitization, continuing exposure to allergens leads to a significant increase in the prevalence of asthma. Ninety percent of children and 80% of adults with asthma are atopic. Once sensitization has occurred, re-exposure to allergen is a risk factor for exacerbations of asthma. Effective management of allergic asthma includes pharmacological therapy and allergen avoidance. The specific physiological effects of asthma associated allergies include airway inflammation, eosinophilia and mucus production, and antigen-specific IgE and IL-4 production. Modulation of Th1 and Th2 subsets, as well as antigen presenting cells, is useful in the treatment of allergic responses. The ability of compounds of this invention to treat asthma can be demonstrated in a murine allergic asthma model according to the method of Wegner et al., Science 247:456-459, 1990, or in a murine non-allergic asthma model according to the method of Bloemen et al., Am J Respir Crit Care Med 153:521-529, 1996.

The ability of compounds of this invention to treat graft rejection can be demonstrated in a murine cardiac allograft rejection model according to the method of Isobe et al., Science 255: 1125-1127, 1992, in a murine thyroid gland kidney capsule model according to the method of Talento et al., Transplantation 55: 418-422, 1993, in a cynomolgus monkey renal allograft model according to the method of Cosimi et al., J Immunol 144: 4604-4612, 1990, in a rat nerve allograft model according to the method of Nakao et al., Muscle Nerve 18:93-102, 1995, in a murine skin allograft model according to the method of Gorczynski and Wojcik, J Immunol 152: 2011-2019, 1994, in a murine corneal allograft model according to the method of He et al., Opthalmol Vis Sci 35:3218-3225, 1994, or in a xenogeneic pancreatic islet cell transplantation model according to the method of Zeng et al., Transplantation 58:681-689, 1994.

The compounds of this invention can be incorporated into a variety of formulations for therapeutic administration. Particularly, agents that modulate ILK activity, or ILK polypeptides and analogs thereof are formulated for administration to patients for the treatment of ILK dysfunction, where the ILK activity is undesirably high or low. More particularly, the compounds of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intracheal, etc., administration. The ILK may be systemic after administration or may be localized by the use of an implant that acts to retain the active dose at the site of implantation.

The compounds of the present invention can be administered alone, in combination with each other, or they can be used in combination with other known compounds. In pharmaceutical dosage forms, the compounds may be administered in the form of their pharmaceutically acceptable salts, or they may also be used alone or in appropriate association, as well as in combination with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

Implants for sustained release formulations are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant is placed in proximity to the site of infection, so that the local concentration of active agent is increased relative to the rest of the body.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Typical dosages for systemic administration range from 0.1 μg to 100 milligrams per kg weight of subject per administration. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

It is to be understood that this invention is not limited to the particular methodology, protocols, cell lines, animal species or genera, constructs, and reagents described, as such may vary. It is also to be understood that the terminology used herein is for the purpose of describing particular embodiments only, and is not intended to limit the scope of the present invention which scope will be determined by the language in the claims.

It must be noted that as used herein and in the appended claims, the singular forms “a”, “and”, and “the” include plural referents unless the context clearly dictates otherwise. Thus, for example, reference to “a mouse” includes a plurality of such mice and reference to “the cytokine” includes reference to one or more cytokines and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for all relevant purposes, e.g., the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Experimental

### EXAMPLE 1

### Neutrophil and Monocyte Accumulation

Acute inflammation is characterized by an initial accumulation of neutrophils (PMN) followed by a tissue accumulation of monocytes (Getting et al. (1997) Br. J. Pharmacol. 120: 1075-1082). It can be advantageous to monitor both processes, which are mechanistically independent one from the other.

Zymosan (an insoluble inflammogen derived from the yeast wall, Ajuebor et al. (1998) J. Leukoc. Biol. 63, 108-116) is injected i.p. in mice, control animals receive only sterile saline. Cellular influx into the peritoneal cavities is evaluated either at the 4 h time-point (for PMN) or at 24 h (for monocytes), by killing animals by CO₂ exposure, and washing the cavities with 3 ml of sterile PBS supplemented with 3 mM EDTA and 25 U/ml heparin. Cells in the lavage fluids are stained in Turk's solution and differential counting is performed with a light microscope.

PMN influx ranges between 15-20 million cells per mouse, and is sensitive to anti-chemokine (i.e. antibodies to MCP-1) and anti-adhesion reagents (i.e. antibody to CD11b) as well as to more classical anti-inflammatory drugs, e.g. PAF antagonists; indomethacin, 5-lipoxygenase inhibitors, etc. The positive control is dexamethasone, which reduces zymosan-induced PMN influx with an approximate ED₅₀ of 0.9 mg/kg s.c. (given 2 h prior to zymosan). The effects of ILK inhibitors are determined in groups pre-treated with various concentrations of ILK inhibitors, usually in systemic administration doses in the range of 10-300 mg/kg, and usually administered every 1-4 hours prior to the cytokine, and up to the time of euthanasia, (dependent on pharmacokinetics of specific ILK inhibitor). Pre-treatment with ILK inhibitors results in a significant decrease in the number of neutrophils that respond to the zymosan stimulus, in turn resulting in significantly lower number of neutrophils infiltrating into the peritoneal cavity.

Monocyte accumulation at the 24 h time-point is in the order of 10-14 million cells per animal. The effect of known drugs on this parameter have not been well investigated, however it is sensitive to anti-chemokine therapy (i.e. antibodies to MCP-1) and to dexamethasone. The latter is the positive control with an approximate ED₅₀ of 0.9 mg/kg s.c. (given 2 h prior to zymosan). The effects of ILK inhibitors are determined in groups pre-treated with various concentrations of ILK inhibitors, usually in systemic administration doses in the range of 10-300 mg/kg, and usually administered every 1-4 hours prior to the cytokine, and up to the 2 hours after (timing dependent on pharmacokinetics of specific ILK inhibitor). Pre-treatment with ILK inhibitors is expected to result in a significant decrease in the number of monocytes that respond to the zymosan stimulus, in turn resulting in significantly lower number of these cells infiltrating into the peritoneal cavity.

TABLE 1

| | | | | PMN |
|---|---|---|---|---|
| | | | Dose | Accumulation |
| | Group | Treatment | (mg/kg) | (× 10⁶ cells) ± SE |
| A | Negative Control | Saline | — | 3.4 ± 0.56 |
| B | Zymosan | Saline | — | 5.6 ± 0.54 |
| C | Zymosan | Dexameth. | 1.5 | 4.0 ± 0.79 |
| D | Zymosan | 31794 | 200 | 5.6 ± 0.92 |
| | | (ILK inhibitor) | | |
| E | Zymosan | 36716 | 200 | 5.2 ± 0.59 |
| | | (ILK inhibitor) | | |
| F | Zymosan | 37178 | 100 | 3.5 ± 0.62 |
| | | (ILK inhibitor) | | |
| G | Zymosan | 47423 | 200 | 5.7 ± 0.79 |
| | | (ILK inhibitor) | | |

Effect of ILK inhibitors on PMN accumulation in mouse peritoneal cavity by Zymosan injection. Mice were pretreated either with Dexamethasone (s.c) or with ILK inhibitors (oral administration) 60 min prior to and 60 min following i.p. injection of Zymosan (1 mg/mouse in 0.5 ml sterile saline). PMN accumulation in peritoneal cavities was measured 4 h later. Data are mean ± SE of n = 5 mice/group.

### EXAMPLE 2

### Cytokine Induced Neutrophil Accumulation

Acute accumulation of neutrophils (PMN) into a specific tissue site is a hallmark of the inflammatory response, and can be reproduced in vivo in the mouse following IL-1β injection into a preformed s.c. dorsal air-pouch. This model has the advantage of a quick (maximal at 4 h) and intense (between 5 and 8 million PMN per animal) response (Perretti & Flower (1993) J. Immunol. 150:992-999).

Six-day-old air-pouches are formed on the back of mice by injection of 2.5 ml of air on day 0 and day 3. On day 6, 5 ng of murine recombinant IL-1β is injected into the air-pouch, or vehicle only, i.e. 0.5% carboxymethylcellulose in PBS. Cellular influx into the air-pouches is evaluated at the 4 h time-point, by killing animals by CO₂ exposure, and washing the cavities with 2 ml of sterile PBS supplemented with 3 mM EDTA and 25 U/ml heparin. Cells in the lavage fluids are stained in Turk's solution and differential counting is done with a light microscope.

This model is not sensitive to indomethacin and BWA4C, but is sensitive to PAF antagonists, anti-adhesion molecule reagents, tachykinin NK1 antagonists, etc. The effects of ILK inhibitors are determined in groups pre-treated with various concentrations of ILK inhibitors in systemic administration doses in the range of 10-300 mg/kg, administered every 1-4 hours prior to the cytokine, and up to the time of euthanasia. Pre-treatment with ILK inhibitors is expected to result in a significant decrease in the number of neutrophils that respond to the IL-1β stimulus, in turn resulting in significantly lower number of neutrophils infiltrating into the air pouch. The positive control is dexamethasone which inhibits IL-1β-induced PMN migration with an approximate ED₅₀ of 0.15 mg/kg s.c. (given 2 h prior to

### EXAMPLE 3

### Chemokine Induced Monocyte Accumulation (Chemokine-Induced)

The use of ‘clean’ experimental models to assess leukocyte migration increases the chances of identifying novel anti-inflammatory drugs acting through specific mechanisms. The recent identification of several selective chemokines responsible for the host inflammatory response for the recruitment of specific leukocyte sub-sets provides an extremely interesting target (Ajuebor et al. (1998) J. Leukoc. Biol. 63, 108-116).

Monocyte chemoattractant protein-1 (MCP-1) given i.p. (1 μg) induces a selective accumulation of monocytes which is maximal at the 6 h post-injection (between 1-3 million monocytes per mouse), above the mild effect of the vehicle (0.25 ml of sterile saline) alone. After MCP-1 injection, animals are killed by CO₂ exposure, and cavities washed with 3 ml of sterile PBS supplemented with 3 mM EDTA. Cells in the lavage fluids are stained in Turk's solution and differential counting is performed using a light microscope by a scientist unaware of the experimental groups. Monocyte influx is also confirmed by staining with F4/80 and FACS analysis. The effects of ILK inhibitors are determined in groups pre-treated with various concentrations of ILK inhibitor in systemic administration doses in the range of 10-300 mg/kg, administered every 1-4 hours prior to the cytokine, and up to the time of euthanasia (timing dependent on pharmacokinetics of specific ILK inhibitor). Pre-treatment with ILK inhibitors is expected to result in a significant decrease in the number of monocytes that respond to the MCP-1 chemokine stimulus, in turn resulting in significantly lower number of these cells infiltrating into the peritoneal cavity. The positive control is dexamethasone which produces approximately 50% inhibition at the dose of 0.1 mg/kg per mouse given s.c. 1 h prior to MCP-1.

### EXAMPLE 4

### Intravital Microscopy

An advanced way to investigate potential inhibitors of leukocyte recruitment is the intravital microscopy technique which allows direct observation of an inflamed vascular bed, (see Islam et al. (1998) Circulation 98(21): 2255-2261).

Mice are injected i.p. with 20 ng mouse recombinant IL-1β and left at liberty until the beginning of the experiment. Control animals receive sterile saline alone (1 ml per rat i.p.). Animals are anaesthetised with Inactin™, shaved on the neck and abdominal areas, and a tracheotomy is performed to facilitate breathing during the experimentation. Cautery incisions are made along the abdominal line, the mesentery is exposed and placed on a viewing plexiglass stage.

The preparation is mounted on a Zeiss Axioskop and is then transilluminated with a 12-V, 100-W halogen light source. Images are acquired by a Hitachi CCD colour camera (model KPC571; Tokyo, Japan) and are displayed onto a Sony Trinitron colour video monitor (model PVM 1440QM) and recorded on a Sony superVHS video cassette recorder (model SVO-9500 MDP) for subsequent off-line analysis. A video time-date generator projects the time, date and stopwatch function onto the monitor.

Particular care is taken to assure observation of the mesenteric vascular bed exactly 2 h after injection of the cytokine. Mesenteries are superfused with thermostated (37° C.) bicarbonate-buffered solution (pH=7.4, gassed with 5% CO₂/95% N₂) at a rate of 2 ml/min. Three to five randomly selected post-capillary venules (diameter between 20-40 μm; length of at least 100 μm) are observed for each rat. Off-line analysis is performed to quantify the extent of cell adhesion (the number of adherent leukocytes in 100 μm length vessel wall), and leukocyte emigration (the number of cells that had emigrated out of the vessel up to 50 μm, 50 to 100 μm and 100-150 μm away from the vessel wall in parallel with 100 μm vessel segments).

IL-1β produces an intense response (>20 adherent cells per 100 μm length) that is modulated in part by endogenous PAF, ICAM-1 and the chemokine CINC. Therefore, agents that interfere with these mediators are identified using this technique. The effects of ILK inhibitors are determined in groups pre-treated with various concentrations of ILK inhibitors, in systemic administration doses in the range of 10-300 mg/kg, and administered every 1-4 hours prior to the cytokine, and up to the 2 hours after (timing dependent on pharmacokinetics of specific ILK inhibitor). Pre-treatment with ILK inhibitors is expected to result in a significant decrease in the number of leucocytes that are recruited.

This technique is applied to other stimuli of leukocyte-endothelium interaction and mesenteries are superfused with the mast cell activator compound 48/80, or with the leukocyte chemoattractants PAF, fMLP or substance P. Peripheral blood is collected and the expression of surface adhesion molecules CD11a and CD11b is evaluated using FACS and WT.1 (Integrin αL, LFA-1α) and WT.5 (Integrin αM, Mac-1α), respectively. Pre-treatment with ILK inhibitors will result in a significant decrease in the expression of adhesion molecules on PB leucocytes.

The positive control is Dexamethasone (given s.c. 1 h prior to the cytokine) which reduces IL-1β-induced cell adhesion with an ED50 of 0.48 mg/kg and IL-1β-induced emigration with an ED50 of 0.04 mg/kg (Tailor et a. (1997) J. Leukoc. Biol. 62: 301-308).

Mouse mesenteric vascular bed was inflamed with interleukin-1β. The anti-inflammatory properties of KP-401-A (an ILK inhibitor) were compared to dexamethasone (positive control) as determined by intravital microscopy. Dexamethasone (0.5 mg/kg) was administered s.c. 1 hour prior to IL-1β (i.p), and 37178 was given orally at 100 mg/kg, both one hour prior and one hour post IL-1β injection. 37178 was formulated in 5% Tween 80. Microscopic observations were made 2 hours following IL-1β injection.

Cell rolling was measured by white blood cell velocity. Cell adhesion was quantified by counting, for each vessel, the number of adherent neutrophils in a 100 μm length. Leukocyte emigration from the microcirculation into the tissue was quantified by counting the number of cells that had emigrated up to 50 μm away from the wall of the 100 μm vessel segments. Oral dosing with 37178 (100 mg/kg) reversed IL-1β mediated blockage of cell rolling, and brought the extent of cell adhesion and emigration back to saline values, thereby completely abrogating the effects of IL-1β. 37178 inhibits the process of leukocyte-endothelium interaction promoted by IL-1β in the mouse mesenteric microcirculation.

TABLE 2
ILK Inhibitors Block Leukocyte-Endothelium Interaction

| | | | | Emigrated |
|---|---|---|---|---|
| | Cell | Cell | Adherent | Cells |
| | Flux | Rolling | Cells | (no. |
| Group | (no. per min) | (V_{WBC}) | (no. 100 μm) | 50 × 100 μm²) |
| Saline | 6.0 ± 1.5 | 22 ± 2.5 | 1.7 ± 0.2 | 1.5 ± 0.4 |
| IL-Iβ + | 14.0 ± 2.0 | 12 ± 3.5 | 4.0 ± 0.2 | 4.5 ± 0.5 |
| DEX + | 7.0 ± 1.5 | 17 ± 2.5 | 1.4 ± 0.1 | 1.8 ± 0.3 |
| Tween 80 + | 17.0 ± 2.0 | 14 ± 4.5 | 3.6 ± 0.3 | 4.0 ± 0.3 |
| 37178 | 8.5 ± 1.5 | 22 ± 2.5 | 1.3 ± 0.2 | 1.0 ± 0.2 |
| 100 mg/kg + | | | | |
| 37178 | 8.0 ± 2.0 | 21 ± 2.0 | 2.5 ± 0.1 | 1.6 ± 0.4 |
| 150 mg/kg | | | | |

What is claimed is:

1. A method of modulating the trafficking of leukocytes in a mammalian host, the method comprising:
administering an effective amount of an integrin linked kinase (ILK) modulating agent, in a dose effective to modulate said trafficking of said leukocytes.

2. The method of claim 1, wherein said administration provides for a prolonged localized concentration of said ILK modulating agent.

3. The method of claim 1, wherein said leukocytes are polymorphonuclear cells.

4. The method of claim 1, wherein said leukocytes are monocytes.

5. The method of claim 1, wherein said host is suffering from an undesirable inflammatory response associated with said leukocytes.

6. The method of claim 1, wherein said ILK modulating agent is an ILK inhibitor.

7. The method according to claim 1, wherein said ILK modulating agent is an ILK agonist.

8. The method according to claim 1, wherein β1 integrin activation is required for extravasation of said leukocytes.

9. The method of claim 1, wherein β3 integrin activation is required for extravasation of said leukocytes.
